(19) United States
(12) Patent Application Publication
(10) Pub. No.: US 2007/0122502 A1
(43) Pub. Date: May 31, 2007

(54) Therapeutic juice composition for women

(76) Inventor: Lawrence M. Logsdon, Laguna Niguel, CA (US)

Correspondence Address: KARAMBELAS & ASSOCIATES, 916 SILVER SPUR ROAD, SUITE 306, ROLLING HILLS ESTATES, CA, 90274, US

(21) Appl. No.: 11/602,126

(22) Filed: Nov. 20, 2006

Related U.S. Application Data
(60) Provisional application No. 60/740,954, filed on Nov. 30, 2005.

(51) Int. Cl. A61K 36/28; A61K 36/87; A61K 31/05; A61K 31/555; A61K 31/715

(52) U.S. Cl. 424/764; 424/777; 424/766; 514/184; 514/736; 514/54

(57) ABSTRACT

A therapeutic juice composition is provided which is particularly advantageous and beneficial to women. This composition comprises noni fruit and a vitamin blend comprising black cohosh, a chromium composition, a calcium composition and a source of trans-resveratrol.

### I. CLAIM OF PRIORITY FROM COPENDING PROVISIONAL PATENT APPLICATION

This application claims priority under 35 U.S.C. 119(e) and 120 from provisional patent application No. 60/740,954, filed on Nov. 30, 2005, the disclosure of which is incorporated herein by reference in its entirety.

### II. FIELD OF THE INVENTION

This invention is directed to therapeutic juice compositions for human consumption and, more specifically, to a therapeutic noni juice composition for women.

### III. BACKGROUND OF THE INVENTION

In today's alternative medicine world, noni fruit enjoys a highly-prized position as a medicinal commodity, just as it was in ancient times. However, modern methods of extracting and preparing noni juice to eliminate much of the unpleasant smell and taste without compromising beneficial properties continue to require further research and development. Additionally, noni is found to produce therapeutic results in aiding health problems such as immune system disorders, bacterial and viral infections, respiratory problems, allergies, arthritis, diabetes, cancer, high blood pressure, menstrual cramps, poor digestion, tuberculosis, malaria, influenza, drug and alcohol addiction, among other afflictions.

The vast therapeutic spectrum of treatments attributable to noni is dramatically witnessed by testimonials from users who have experienced dramatic results. Numerous doctors have documented that countless patients have been assisted by the noni juice. Scientific studies have shown that noni has the potential to stimulate the immune system, inhibit tumor growth, regulate proper cell function, purify the blood, and regenerate damaged cells, among other properties. These broad-ranging results have inspired researchers to continue to explore and develop better methods of producing, harvesting, transporting, purifying and blending this fruit and compositions which optimize its therapeutic benefits.

Modern day drugs used to treat illnesses and diseases are generally referred to as pharmaceuticals. Phytochemicals in plants having potential health benefits are referred to an nutraceuticals, of which noni is found to contain a variety of beneficial phytochemicals, some of which are not found in any other known plant. These phytochemicals work synergistically to enhance biologically the expression of each individual component found in noni so that scientists are incentivized to optimize these therapeutic qualities of noni to dramatically improve its disease preventing and health promoting benefits.

Noni (morinda citrifolia) is said contain over 150 nutraceuticals, among which are the following: Xeronine, Scopoletin, Proxeronine, Morindadiol, Proxeronase, Rubiadin, Serotonin, Magnesium, Damnacanthal, Carbonate Nordamnacanthal, Protein, Antraquinones, Sodium, Carotenoids, Bioflavinoids, Morindine, Morindone, Terpenes, Plant Sterols, Iron, Sitosterol, Phosphate, Glycosides, Carbohydrates, Aleizarin, Acetin, GlucoP, Ursolic acid, Caproic acid, Caprlyic, Glucopyranose, Asperuloside, Precursors, Vitamins, Trace elements, Alkaloids, Enzymes, Serine, Multi-receptor activators, Chlororubin, Methionine, Alanine, Isolucine, Arginine, Leucine, Aspartate, Lysine, Cysteine, Phenlyalanine, Cystine, Threonine, Glycine, Trypophane Glutamate, Valine, Tyrosine, Histadine, and Proline.

Among these noni possesses a rich source of Proxeronine, Proxeronase and Xeronine, which are alkaloids employed by the body to strengthen and revitalize cells, as well as possessing a rich source of antioxidants and essential nutrients.

Consequently, substantial research has been performed worldwide by modern scientists that have led them to acknowledge noni's unique healing properties through its high quantities of phytonutrients and enzymes. Beginning in the 1950s, noni has been researched at leading institutions in France, the Netherlands, Germany, Ireland, Taiwan, Japan, Austria, Canada and Hawaii.

In 1993 a Japanese research team reported in the Cancer Letters that noni was the most effective of 500 tropical plant extracts at preventing the growth of tumors. Studies have shown that noni stimulates the production of T-cells in the immune system. Other studies have shown noni to have very effective anti-inflammatory analgesic antibacterial properties. One study at the University of Hawaii isolated one of the 140 compounds, scopoletin, and demonstrated that it lowered high blood pressure, relaxed spasms, reduced inflammation, killed bacteria and fungi and prevented growth of tumors. Two reasons are postulated by scientists for noni's wide range of effectiveness are its ability to regulate cell function and cellular regeneration and its role as an adaptogen; i.e., helping the body to restore its organs to normal function or homeostasis.

Typical improvements when ingesting noni are illustrated in the following chart:

| | No. | | |
|---|---|---|---|
| | Taking | | |
| | Noni | | Primary |
| Condition Reported To | for | Percent | Active |
| Respond to Noni | Condition | Improvement | Component |
| Allergy, improved symptoms | 948 | 84% | Xeronine |
| Anti-Aging, look better | 148 | 78% | Terpene |
| Arthritis, lessened symptoms | 719 | 81% | Terpene |
| Breathing, improved | 2,854 | 77% | Xeronine |
| Cancer, reduced symptoms | 889 | 65% | Xeronine |
| Depression, lessened symptoms | 807 | 77% | Xeronine |
| Diabetes, Types I and II | 2,773 | 84% | Scopoletin |
| Digestion improved | 1,593 | 89% | Xeronine |
| Energy Increased | 8,327 | 92% | Xeronine |
| Fuzzy thinking, helped clear | 373 | 88% | Xeronine |
| Heart Disease, improved | 1,123 | 80% | Xeronine |
| symptoms | | | |
| High Blood Pressure, | 938 | 85% | Scopoletin |
| improved | | | |
| Kidney health, improved | 2,372 | 67% | Xeronine |
| Mental acuity, increased | 2,983 | 72% | Xeronine |
| alertness | | | |
| Muscle, increased body- | 816 | 71% | Protein |
| building | | | |
| Obesity, lost excess weight | 2,841 | 75% | Xeronine |
| Pain, including headaches, | 4,231 | 88% | Xeronine |
| improved | | | |
| Sexuality, enhanced enjoyment | 1,608 | 87% | Xeronine |
| Sleep, improved | 1,231 | 73% | Xeronine |
| Smoking, stopped | 452 | 58% | Xeronine |
| Stress, helped cope with | 4,113 | 72% | Xeronine |
| Stroke | 1,019 | 57% | Xeronine |
| Increased feeling of well being | 4,561 | 80% | Xeronine |

The literature is likewise rife with testimonials and anecdotal evidence of the effectiveness of the noni plant.

Thus there is seen to be a dramatic need for utilizing and optimizing the benefits of the noni plant by employing compositions to further enhance and synergize the therapeutic aspects of noni.

In U.S. Pat. No. 7,070,813, filed Oct. 31, 2002, issued Jul. 4, 2006, there is disclosed methods and formulations or compositions for the treatment of colon cancer and particularly for the inhibition, prevention and/or reduction of cancerous cell growth as well as the destruction of early stage cancerous cells within the colon region of a mammal, wherein the formulations and compositions comprise an identified amount or concentration of a processed morinda citrifolia product or an active ingredient therefrom as obtained from the Indian mulberry plant.

In U.S. Pat. No. 6,528,053, filed Feb. 12, 2002, issued Mar. 4, 2003, there is taught compositions and methods for reducing oxysterol buildup in the blood and normalizing cholesterol and blood pressure in a mammal employing compositions comprising morinda citrifolia extract, red wine extract, prune extract, blueberry extract, pomegranate extract, apple extract, and an enzyme mixture.

In U.S. Pat. No. 6,440,410, filed Nov. 28, 2001, issued Aug. 27, 2002, there is disclosed compositions and methods for reducing oxysterol buildup in the blood and normalizing cholesterol and blood pressure in a mammal employing compositions that comprise morinda citrifolia extract, red wine extract, prune extract, blueberry extract, pomegranate extract, apple extract, and an enzyme mixture.

In U.S. Pat. No. 6,436,449, filed Mar. 2, 2001, issued Aug. 20, 2002, there is disclosed the use of an extract derived from the fruits, leaves, the bark or the roots of morinda citrifolia L. for the manufacture of a medicament for the treatment of a mammal suffering from tinnitus. The extract may be a liquid present in the medicament in an amount such as to give a daily dosage of 0.1-2 ml, or 0.2-1 ml, e.g. 0.4-0.7 ml, per kg body weight of the patient. The extract also may be a solid present in the medicament in an amount such as to give a daily dosage of 5-200 mg, or 10-100 mg, e.g. 20-70 mg, per kg body weight of the patient. Optionally, the medicament also may comprise lycopene, vitamin C, coenzyme Q10 and an extract from the leaves of Ginkgo biloba. The medicament may be given e.g. by oral, rectal, transdermal or inhalation administration.

In U.S. Pat. No. 6,403,086, filed Nov. 30, 2001, issued Jun. 11, 2002, there is disclosed compositions and methods for reducing oxysterol buildup in the blood and normalizing cholesterol and blood pressure in a mammal employing compositions comprising morinda citrifolia extract, red wine extract, prune extract, blueberry extract, pomegranate extract, apple extract, and an enzyme mixture.

In U.S. Pat. No. 6,387,370, filed Jan. 19, 2001, issued May 14, 2002, there is disclosed compositions and methods for reducing oxysterol buildup in the blood and normalizing cholesterol and blood pressure in a mammal employing compositions comprising morinda citrifolia extract, red wine extract, prune extract, blueberry extract, pomegranate extract, apple extract, and an enzyme mixture.

In U.S. Pat. No. 5,288,491, filed Sep. 24, 1992, issued Feb. 22, 1994, there is disclosed a method for processing the noni plant into powder including the steps of picking the noni fruit from the tree, placing picked noni fruit in a room, washing and cleaning the noni plant, mashing the noni fruit, placing the pulp onto liner, rotating trays for five hours, rotating trays for another five hours, rotating trays for another 14 hours, and crushing and grinding dried wafers.

In U.S. Pat. No. 6,589,514, filed Apr. 17, 2001, issued Jul. 8, 2003, there is disclosed repair serums comprising morinda citrifolia from the Indian mulberry plant, providing significant skin care advantages not found in prior art intensive repair serums.

In U.S. Pat. No. 6,855,345, filed Nov. 1, 2002, issued Feb. 15, 2005, there is disclosed a unique natural formulation and method of administering the same to treat and prevent diabetes, or rather advances treatment of diabetes, by providing a naturaceutical composition or treatment formulated with one or more processed morinda citrifolia products as derived from the Indian mulberry plant. The morinda citrifolia is particularly adapted to treat Type II diabetes. The morinda citrifolia product is preferably a leaf extract, but may also be in the form of a juice, a puree juice, a dietary fiber, or other similar forms and is incorporated into various carriers or naturaceutical compositions suitable for in vivo treatment of a patient. The naturaceutical may also combine other food products into the naturaceutical, such as fruit juices, dietary supplements, vitamins and minerals, and others.

It is known that females, upon reaching middle age or as early as the age of 35, who suffer from various ailments, including but not limited to, hot flashes, night sweats, mood swings, insomnia and fatigue, which are the more noticeable changes in their quality of life. These changes have been generally attributed to hormone deficiencies and are also associated with changes which are not generally as noticeable as the aforementioned changes in life quality including, but not limited to, bone thinning, increased cholesterol, high blood sugar and free radical change to the body's cells that can prove to be most dangerous contributing to heart disease, osteoporosis, premature aging, arthritis, diabetes and other disorders.

Prior art solutions to these maladies generally speaking include taking hormones or hormone therapy which is now considered, in addition to being ineffective, dangerous to a woman's quality of life.

There is, therefore, a demonstrated need for treatment of these female maladies experienced at the onset of middle age.

### IV. OBJECTS OF THE INVENTION

It is therefore an object of this invention to replace dangerous hormone therapy with a safe and natural juice composition to treat maladies experienced at the onset of middle age in females.

It is a further object of this invention to provide a noni juice therapeutic composition which allows a female to maintain a good quality of life and eliminate maladies associates with the onset of middle age.

A further object of this invention is to provide a noni juice therapeutic composition to treat hot flashes, night sweats, mood swings, insomnia and fatigue associated with the onset of middle age in females.

Yet another object of this invention is to provide a noni juice therapeutic composition to treat bone thinning, increased cholesterol, high blood sugar and free radical damage to the body's cells which is experienced by females at the onset of middle age.

Yet again another object of this invention is to provide a noni fruit juice composition combined with high powered natural ingredients which assists in the balancing of hormones and improves bone health and provides protection of cardiac function.

Still another object of this invention is to provide a therapeutic juice composition to treat maladies associated with the onset of middle age in females devoid of the above noted deficiencies.

### V. BRIEF SUMMARY OF THE INVENTION

These and other objects of the instant invention are accomplished generally speaking by providing a therapeutic noni fruit juice composition to treat maladies associated with the onset of middle age in females.

The therapeutic fruit juice composition of the instant invention comprises noni fruit (Morinda citrifolia) and a vitamin blend comprising black cohosh, ChromeMate®, a scientifically designed form of chromium, calcium L-threonate, and a source of trans-resveratrol.

It is found that this therapeutic noni juice composition which represents a highly fortified version of the noni juice composition, commercially referred to as Bio-Noni® for Women, specifically targets the above recited female problems and is conveniently provided in a liquid form wherein two tablespoons of Bio-Noni® are found to contain a surprisingly high concentration of 10,000 milligrams of noni extract.

This combination of natural botanicals and scientific extracts provides relief for women from the above recited maladies brought on at the onset of middle age and other menopausal symptoms.

### VI. DETAILED DESCRIPTION OF THE INVENTION

The noni fruit (Morinda citrifolia) in its preferred and most effective form is found in Tahiti and has been used for more than 2,000 years as a natural painkiller and anti-inflammatory. This fruit which comprises a botanical medicine is employed to treat allergies, obesity, menstrual cramps, arthritis and related conditions and has enjoyed the endorsement of an increasing number of mainstream physicians and medical researchers.

Black cohosh (Cimicifuga racemosa) has been employed by Native Americans to treat “female problems” for centuries. Today, hundreds of studies have confirmed that black cohosh is just as effective as estrogen in relieving menopausal hot flashes and night sweats while easing the mood swings, irritability and sleeplessness that accompany them. Although it is not known how black cohosh actually works, one theory that has been offered is the ability of this ingredient to slow down the action of lutenizing hormone (LH) which causes the ovaries to release an egg. It is known that as menopause approaches, more and more LH is released in order to have the ovaries release an egg. These ever-increasing amounts of LH may be responsible for interfering with the brain's temperature control area, resulting in episodes of heat to the face and neck. Consequently, it is found by dampening the action of LH employing black cohosh, these hot flashes are attenuated.

The mineral chromium is one of the body's greatest unrecognized housekeepers which plays a critical role in sending nutrition to the cells, metabolizing fat and turning protein into muscle. In addition, it helps regulate the appetite, increase lean body mass and reduces sugar cravings. Another attribute of chromium is that it also lowers LDL, which is referred to as the bad cholesterol, and raises HDL, referred to as the good cholesterol helping to protect the heart.

The typical American diet, especially for females, is low in chromium and further exacerbating the situation it is found that the body absorbs less and less of this important mineral as it ages. The body's storage of chromium can be diminished by stress, infection, physical trauma and a diet high in refined foods. As this occurs, a subject female is more likely to suffer from poor insulin function, Type II diabetes, high levels of cholesterol and blood fat, overweight, heart disease and a general lack of energy.

The remedy of employing a chromium supplement is not an easy solution since chromium is poorly absorbed and not easily converted to its active form in order to remedy the above recited deficiencies. It is found, however, that when chromium is combined with niacin in the form of chromium nicotinate, commercially available as ChromeMate®, it is absorbed and retained by the body six times better than standard chromium supplements. This commercial adaptation of chromium composition is also superior in enhancing insulin sensitivity, lowering blood pressure and fighting free radicals to help ward off diabetes, overweight, heart disease, premature aging and other degenerative diseases.

With the onset of menopause, there can also be a noticeable loss in females of bone mass which may even be realized at 2 or even 3% per year or more. Alarmingly, this may increase even at minimal rates to approximately a 50% loss of original bone mass by age 70 resulting in ease of bone fracturing, collapsed vertebrae, humped spine, broken hips, in addition to a general diagnosis of osteoporosis.

The lack of calcium, especially in a female's diet, is generally the cause of these maladies. However, taking calcium as a supplement results in only 5-10% of most calcium supplements being actually absorbed by the body. Surprisingly, it is found that the use of calcium L-threonate (provided commercially in the form known as BioCalth®) results in an amazing 95% absorption rate by the body. In addition, BioCalth® has two other attributes that no other calcium supplement provides, significantly increases bone mass by stimulating the building of new bone tissue and enhancing the production of collagen, a major structural ingredient in both bone and cartilage. Generally speaking, the BioCalth® formulation provides the body with a basis to rebuild both bone tissue and joint cartilage in a high absorbable form.

Low estrogen levels are also associated with hot flashes, vaginal dryness and high cholesterol, whereas higher than normally acceptable levels of estrogen are found to cause depression, irritability and bloating. Compositions containing trans-resveratrol, a potent phytoestrogen, commercially available as Protykin®, polygonum cuspidatum, which comprises an extract taken from Chinese herbs are found most useful in balancing estrogen levels to avoid these complications.

Current research suggests that trans-resveratrol, which is found in grapes, may be even more effective than soy or red clover at reducing hot flashes, balancing mood swings, maintaining bone density, promoting cardiovascular health and preventing premature aging. Thus it is found that compositions containing trans-resveratrol are effective in rebalancing hormones in order to provide a better quality of life in females at the onset of middle age.

Although each of these substituents in the therapeutic noni juice composition of the instant invention are found to provide significant properties associated with the individual characteristics of these substituents, an effective combination of noni juice, black cohosh, chromium element composition, calcium L-threonate and polygonum cuspidatum extract (a source of trans-resveratrol) has surprisingly synergistic and superior properties than the individual substituents from which it is comprised, providing a liquid composition that surprisingly provides superior therapeutic effects to the female user, including but not limited to, alleviating hot flashes and mood swings; normalizing estrogen balance; fighting depression and fatigue; relieving tension and anxiety; promoting a feeling of overall well-being; promoting cardiovascular health; promoting immune system function; aiding in management of healthy blood sugar levels; aiding in maintaining healthy body weight; providing super-antioxidant protection; building bone strength and density; stimulating collagen production; and promoting joint and cartilage health. All of these attributes harness the synergistic combination of these substituents to provide superior and surprising results which are far greater than those attributed to the individual respective substituents comprising the composition.

Any suitable noni juice may be employed in the therapeutic juice composition of the instant invention. Typical noni juices include those from the Hawaiian Islands, Tahiti and elsewhere in Polynesia. Preferred of these is the Tahitian Noni Juice. When employed in extract form, it comprises a solution containing noni fruit solids and water at a brix of about 6.0 or greater.

Any suitable black cohosh may be employed in the therapeutic juice composition of the instant invention. Typical black cohosh substituents are known by those skilled in the art as commercially available.

Any suitable source of mineral chromium may be employed in the therapeutic juice composition of the instant invention. Typical compositions containing chromium include powder and tablet supplements containing chromium associated with other minerals and chromium in combination with niacin. Preferred of these is chromium in combination with niacin available commercially in the form of ChromeMate®.

Any suitable source of calcium may be employed in the therapeutic fruit juice composition of the instant invention. Typical forms of calcium include those in powder and tablet form associated with other minerals and calcium L-threonate. Preferred of these is calcium L-threonate commercially available as BioCalth®.

Any suitable composition providing trans-resveratrol, polygonum cuspidatum, may be employed in the therapeutic juice composition of the instant invention. Typical materials containing trans-resveratrol include Chinese herbs, soy, red clover and grapes. Preferred of these is the trans-resveratrol contained in grapes.

In addition to the noni juice, black cohosh, chromium (ChromeMate®) compositions, calcium (BioCalth®), trans-resveratrol (Protykin), other ingredients may be added to render the resulting liquid elixir more palatable to the taste and more pleasing to the senses. Filtered water is employed to obtain the proper viscosity and flowing characteristics of the liquid. In addition, xanthan gum, fructose, commercially available as Fructose 42 high fructose corn syrup (HFCS) having a brix of about 71°, the commercially available concord grape juice concentrate, a commercially available white grape juice concentrate, a commercially available raspberry fruit extract, a commercially available concord grape juice, a commercially available exberry grape blue stable providing blue color, and sufficient citric acid to result in about a ph of 3.5.

In addition, a proprietary vitamin blend may be employed which provides, in addition to the Protykin KG, ChromeMate®, BioCalth® and black cohosh, synergistically effective amounts of vitamin B12, vitamin B6, folic acid, magnesium and L-tyrosine to result in a highly preferable commercial composition available as Tahiti Trader's® Bio-Noni® for Women.

Although any suitable percentage of the above recited ingredients may be employed to provide an effective therapeutic juice composition for women, typical weight compositions of the above ingredients include water percentage by weight of 40-60%, xanthan gum of 0.09-0.11%, noni extract of approximately 25-50%, fructose of 1-3%, concord grape of 1-3% (68° brix), white grape of 2-4%, citric acid at 0.5-2%, concord grape at 0.1-0.4%, vitamin blend of 0.9-1.1%, exberry grape extract 0.05-0.2%.

Although the proprietary vitamin blend may comprise any suitable combination of ingredients in varying weight percentages, a typical vitamin blend may comprise: Protykin KG at about 0.05-0.002%, ChromeMate® at about 10-30%, BioCalth® at about 0.1-0.4%, black cohosh 0.005-0.02%, vitamin B12 0.02-0.05%, vitamin B6 0.0002-0.0006%, folic acid 0.00001-0.00002%, magnesium 20-50%, and L-tyrosine 20-50%.

A preferred composition would include filtered water at about 50%, xanthan gum at about 0.1%, noni extract at about 38%, fructose HFCS at about 2.3%, concord grape at about 2.2%, white grape at about 2.7%, natural raspberry flavor at about 1.2%, concord grape flavor at about 0.26%, Tahiti Trader's® noni powder at about 0.4%, proprietary vitamin blend at about 1%, and exberry grape blue stable coloring at about 0.1%, wherein the proprietary vitamin blend comprises Protykin KG at about 0.001%, ChromeMate® at about 20%, BioCalth® at about 0.2%, black cohosh at about 0.02%, vitamin B12 at about 0.04%, vitamin B6 at about 0.004%, folic acid at about 0.00002%, magnesium at about 40%, and L-tyrosine at about 40%.

These compositions are extremely beneficial to all women, but particularly for those women over the age of about 35. For these women, Bio-Noni® for Women in the above recited composition is found to provide an all natural anti-aging supplement for the relief of PMS or menopause symptoms quickly, effectively and safely while building bone density and providing cardio-vascular benefits. In addition to preventing the effects of premature aging, it is effective in assisting the control of mood swings, irregular cycles, water retention, hot flashes, night sweats, weight gain, low energy, depression, loss of bone density and effective in minimizing bruising and wrinkling of skin.

In small dosages taken daily, it is a normal and natural method to normalize estrogen balance, fight depression and fatigue, relief tension and anxiety, promote a feeling of well-being, promote cardio-vascular health and immune system function, assist in maintaining healthy blood sugars and healthy body weight, provide super antioxidant protection, building bone strength and density, stimulate collagen production and promote joint and cartilage health.

In doing so, it is found to balance PMS (pre-menstrual symptoms), balance menopause symptoms, promote skin elasticity while reducing wrinkling, promote safe insulin levels by modulating blood sugars, improve fat metabolism and consequently reduce cholesterol and triglyceride levels, assist in maintaining lean muscle tissue and proper body weight, assist in promoting healthy immune system function and provide cardiovascular antioxidant protection.

The compositions of the instant invention go to work immediately to balance a women's system naturally and safely without the negative side effects of common HRT (hormone replacement therapy) and act as a natural plant form of estrogen that actually mimics a female's naturally occurring estrogen.

The black cohosh extract substituent, in combination with the other substiuents recited above, synergistically assists in reducing hot flashes, mood swings, night sweats, irritability and occasional sleeplessness.

The above recited formulations, in addition, contain a calcium composition commercially known as BioCalth® which has been shown to assist in building bone strength, density and enhance cartilage repair function, stimulate and assist in collagen and bone formation while maintaining proper joint function and mobility.

The Bio-Noni® for Women compositions of the instant invention act as a natural plant estrogen that works together to promote cardiovascular health and antioxidant protection. The above recited Bio-Noni® for Women compositions with the above recited natural plant estrogen properties, together with an all natural bioactive form of chromium, is seen to protect the heart from free-radical damage and regulates blood sugars to help maintain healthy insulin levels.

The Bio-Noni® for Women composition of the instant invention may be prepared employing conventional mixing apparatus and added to same in any suitable order.

While the present invention has been particularly described with respect to certain substituent elements in its preferred embodiment, it will be understood that the invention is not limited to these particular substituents described in the preferred embodiments, or the sequence in adding substituents or processing the substituents. On the contrary, it is intended to cover all alternatives, modifications, and equivalents as may be included within the spirit and scope of the invention defined by the appended claims.

In addition, other substituents may be employed in the compositions of the instant invention as claimed as well as variations and alternatives to the methods disclosed and claimed with similar results with regard to the therapeutic characteristics of these compositions. In particular, the scope of the invention is intended to include, for example, those substituents which further enhance and synergize the salutary effects of these compositions, in addition to other substituents which provide further synergy to these compositions.

What is claimed is:

1. A therapeutic juice composition comprising the juice of the noni fruit and a vitamin blend comprising black cohosh, chromium composition, a calcium composition, and a source of trans-resveratrol.

2. The composition as defined in claim 1, wherein said noni fruit is present in a weight percentage of 25-50%, and said vitamin blend is present in a weight percentage of 0.9-1.1%.

3. The composition as defined in claim 1, further comprising water in a weight percentage of 40-60%.

4. The composition as defined in claim 1, further comprising xanthan gum present in a weight percentage of 0.09-0.11%.

5. The composition as defined in claim 1, further comprising fructose present in a weight percentage of 1-3%.

6. The composition as defined in claim 1, further comprising concord grape present in a weight percentage of 1-3%.

7. The composition as defined in claim 1, further comprising white grape present in a weight percentage of 2-4%.

8. The composition as defined in claim 1, further comprising citric acid present in a weight percentage of 0.5-2%.

9. The composition as defined in claim 1, further comprising concord grape present in a weight percentage of 0.1-0.4%.

10. The composition as defined in claim 1, further comprising exberry grape extract present in a weight percentage of 0.05-0.2%.

11. The composition as defined in claim 1, wherein said vitamin blend comprises trans-resveratrol present in a weight percentage of 0.05-0.002%.

12. The composition as defined in claim 11, wherein said chromium composition is present in a weight percentage of 10-30%.

13. The composition as defined in claim 11, wherein said calcium composition is present in a weight percentage of 0.1-0.4%.

14. The composition as defined in claim 11, wherein said trans-resveratrol is present in a weight percentage of about 0.05-0.002%.

15. The composition as defined in claim 11, further comprising vitamin B12 present in a weight percentage of 0.02-0.05%, vitamin B6 present in a weight percentage of 0.0002-0.0006%, folic acid present in a weight percentage of 0.0001-0.00002%; magnesium present in a weight percentage of 20-50%, and L-tyrosine present in a weight percentage of 20-50%.

16. A therapeutic juice composition comprising the juice of the noni fruit and a vitamin blend comprising black cohosh, chromium composition, a calcium composition, and a source of trans-resveratrol, and further comprising concord grape, white grape, concord grape and exberry grape extract.

17. The composition as defined in claim 16, further comprising xanthan gum, fructose and citric acid.
